(19) United States
(12) Patent Application Publication
(10) Pub. No.: US 2024/0293055 A1
(43) Pub. Date: Sep. 5, 2024

(54) Capillary Blood Collection Device

(71) Applicant: Becton, Dickinson and Company, Franklin Lakes, NJ (US)

(72) Inventors: Anthony V. Torris, Mercer Island, WA (US); Kishore K. Bokka Srinivasa Rao, Ridgewood, NJ (US); Scott Wentzell, Suffern, NY (US); Vlad Yakhnich, Park Ridge, NJ (US)

(21) Appl. No.: 18/572,964

(22) PCT Filed: Jun. 23, 2022
(86) PCT No.: PCT/US22/34619

§ 371 Date: Dec. 21, 2023

Related U.S. Application Data
(60) Provisional application No. 63/216,223, filed on Jun. 29, 2021.

(51) Int. Cl. A61B 5/15; A61B 5/151

(52) U.S. Cl. CPC A61B 5/150022; A61B 5/150068; A61B 5/150259; A61B 5/150343; A61B 5/150748; A61B 5/151

(57) ABSTRACT

A device for obtaining a blood sample may include a holder for receiving a sample source, the holder having an actuation portion and a port; a container engagement portion connected to the holder; and a collection container removably connectable to the container engagement portion, the container defining a collection cavity, wherein the actuation portion comprises at least two wings that are angled relative to a patient's finger held in the holder to create a pressure gradient towards a fingertip of the patient's finger.

### CROSS-REFERENCE TO RELATED APPLICATION

The present application claims priority to U.S. Provisional Application Ser. No. 63/216,223, filed Jun. 29, 2021, entitled “Capillary Blood Collection Device”, the entire disclosure of which is hereby incorporated by reference in its' entirety.

### BACKGROUND OF THE INVENTION

### Field of the Invention

The present disclosure relates generally to a device for obtaining a biological sample. More particularly, the present disclosure relates to an integrated finger-based capillary blood collection device with the ability to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medial field is capillary blood collection which is often done to collect blood samples for testing. Certain diseases, such as diabetes, require that the patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member such as a needle, or a sharp-edged member such as a blade, that is used to make a quick puncture wound or incision in the patient's skin in order to provide a small outflow of blood. It is often physiologically and psychologically difficult for many people to prick their own finger with a hand-held needle or blade. As a result, lancet devices have evolved into automatic devices that puncture or cut the skin of the patient upon the actuation of a triggering mechanism. In some devices, the needle or blade is kept in a standby position until it is triggered by the user, who may be a medical professional in charge of drawing blood from the patient, or the patient himself or herself. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releasing the lever from interference engagement with the lancet structure. The blood sample that is received is then collected and/or tested. This testing can be done by a Point-of-Care (POC) testing device or it can be collected and sent to a testing facility.

Currently, capillary blood collection workflow is a complex multi-step process requiring high skill level. The multi-step nature of this process introduces several variables that could cause sample quality issues such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

Capillary collection blood draws are typically performed by health care workers either using their fingers to manually squeeze the tissue around the puncture site or by a device using vacuum pressure to pull blood from the site.

Manually squeezing the collection site is a highly technique dependent process that leads to very large variation in success rate and sample quality (as measured by hemolysis—blood cell rupture). Health care workers typically adjust the pressure and rate at which they squeeze to compensate for patient-dependent differences in blood flow. Squeezing harder helps blood flow more quickly but also increases hemolysis. The location of squeezing also varies between health care workers depending on personal preference, experience, and hand fatigue. Some workers may even perform a process called “milking” of fingers, where they apply pressure starting at the base of the finger and slide towards the tip of finger. This process is discouraged as leading to poor sample quality by domestic and international health organizations.

Vacuum-powered devices standardize the pressure and technique of blood flow, but are typically plagued by poor overall blood flow. The maximum pressure than can be applied is limited by the difference between atmospheric pressure and absolute vacuum (˜14 psi), and devices only operate at a fraction of absolute vacuum. For reference, grip strength of men and women range from 50-100 lbs. on average, illustrating why manual methods are instead affected by hemolysis rather than flow. Vacuum methods also apply consistent pressure, limiting the ability of the tissue to replenish with blood.

Thus, there is a need in the art for a device that has the ability to lance and squeeze the finger, collect the sample, stabilize the sample, and subsequently dispense the sample in a controlled manner. There is also a need in the art for a device that simplifies and streamlines the capillary blood collection by eliminating workflow variabilities which are typically associated with low sample quality including hemolysis and micro-clots. There is still a further need in the art for a closed system collection and transfer that eliminate blood exposure and device reuse. There is still a further need in the art for a device that: (1) introduces flexibility in the accommodation of different capillary blood collection and transfer container; (2) has the capability to generate high quality uniformly mixed/stabilized capillary blood samples; (3) has the capability to generate on-board plasma from capillary plasma samples; (4) has the capability to collect large capillary blood samples (>50-500 μL) at reduced pain; (5) contains a unique sample identifier that is paired with patient information at the time of collection; (6) has the capability to collect capillary blood and perform on-board diagnostics; and (7) has multiple collection ports to collect a blood sample into different containers having the same or different anticoagulants. There is a further need in the art for a capillary blood collection device that includes a standardized and controlled location of applied pressure, an applied pressure that is high enough for adequate blood flow but below hemolysis thresholds, a defined rhythmic application of pressure rather than consistent pressure to allow blood to replenish in the finger, increasing average blood flow rate, and a reduced user fatigue by lowering maximum applied force by the operator.

### SUMMARY OF THE INVENTION

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

In one embodiment of the present disclosure, a device for obtaining a blood sample may include a holder for receiving a sample source, the holder having an actuation portion and a port; a container engagement portion connected to the holder; and a collection container removably connectable to the container engagement portion, the container defining a collection cavity, wherein the actuation portion comprises at least two wings that are angled relative to a patient's finger held in the holder to create a pressure gradient towards a fingertip of the patient's finger.

In one embodiment of the present disclosure, the at least two wings may be positioned on the holder at a position proximal of a fingernail of the patient's finger and distal from a first knuckle of the patient's finger. Each of the at least two wings may include a touch pad that ensures a user squeezes the wings at a desired location. The holder may include a stability extension portion provided on a proximal end of the holder. The stability extension portion may include at least one retention bump that extends inwardly from an inner surface of the stability extension portion to contact the patient's finger held in the holder. At least one wing of the at least two wings may include an anti-touch protrusion that extends outwardly from an outer surface of the wings. The anti-touch protrusion may be positioned beneath a touch pad of the at least one wing to ensure a user grips the at least one wing at the touch pad and not beneath the touch pad. A proximal end of the holder may include a flared edge to receive the patient's finger. A transition portion may be positioned between the holder and the container engagement portion to receive at least a portion of the patient's finger during use of the device. A distal end of the holder may include a curved edge to receive a patient's fingertip while allowing the patient's fingernail to extend past the distal end of the holder. The holder may include a finger receiving portion that includes at least one bulge on an outer surface of the finger receiving portion to prevent pressure on an arterial blood supply of the patient's finger held in the finger receiving portion.

In one embodiment of the present disclosure, a device for obtaining a blood sample may include a holder for receiving a sample source, the holder having an actuation portion and a port; and a container engagement portion connected to the holder; wherein the actuation portion comprises at least two wings that are angled relative to a patient's finger held in the holder to create a pressure gradient towards a fingertip of the patient's finger.

In one embodiment of the present disclosure, the at least two wings may be positioned on the holder at a position proximal of a fingernail of the patient's finger and distal from a first knuckle of the patient's finger. Each of the at least two wings may include a touch pad that ensures a user squeezes the wings at a desired location. The holder may include a stability extension portion provided on a proximal end of the holder. The stability extension portion may include at least one retention bump that extends inwardly from an inner surface of the stability extension portion to contact the patient's finger held in the holder. At least one wing of the at least two wings may include an anti-touch protrusion that extends outwardly from an outer surface of the wings. The anti-touch protrusion may be positioned beneath a touch pad of the at least one wing to ensure a user grips the at least one wing at the touch pad and not beneath the touch pad. A proximal end of the holder may include a flared edge to receive the patient's finger. A transition portion may be positioned between the holder and the container engagement portion to receive at least a portion of the patient's finger during use of the device. A distal end of the holder may include a curved edge to receive a patient's fingertip while allowing the patient's fingernail to extend past the distal end of the holder. The holder may include a finger receiving portion that includes at least one bulge on an outer surface of the finger receiving portion to prevent pressure on an arterial blood supply of the patient's finger held in the finger receiving portion.

In one embodiment of the present disclosure, a device for obtaining a blood sample may include a holder for receiving a sample source, the holder having an actuation portion and a port; a container engagement portion connected to the holder; a collection container removably connectable to the container engagement portion, the collection container defining a collection cavity; and a lancet device removably connected to the container engagement portion, wherein the actuation portion comprises at least two wings that are angled relative to a patient's finger held in the holder to create a pressure gradient towards a fingertip of the patient's finger.

In one embodiment of the present disclosure, a method of operating a device for obtaining a blood sample may include the steps of providing the device for operation by a user, the device comprising: a holder for receiving a sample source, the holder having an actuation portion and a port; and a container engagement portion connected to the holder; wherein the actuation portion comprises at least two wings that are angled relative to a patient's finger held in the holder to create a pressure gradient towards a fingertip of the patient's finger; squeezing the at least two wings together to create the pressure gradient on the patient's fingertip, wherein the at least two wings are squeezed between 0.25 seconds and 1 second; holding the at least two wings closed together up to 0.5 seconds; releasing pressure on the at least two wings up to 0.5 seconds; and holding open the at least two wings up to 1 second.

The present invention is also described in the following clauses:

Clause 1: A device for obtaining a blood sample, the device comprising: a holder for receiving a sample source, the holder having an actuation portion and a port; a container engagement portion connected to the holder; and a collection container removably connectable to the container engagement portion, the container defining a collection cavity, wherein the actuation portion comprises at least two wings that are angled relative to a patient's finger held in the holder to create a pressure gradient towards a fingertip of the patient's finger.

Clause 2: The device of Clause 1, wherein the at least two wings are positioned on the holder at a position proximal of a fingernail of the patient's finger and distal from a first knuckle of the patient's finger.

Clause 3: The device of Clause 1 or 2, wherein each of the at least two wings includes a touch pad that ensures a user squeezes the wings at a desired location.

Clause 4: The device of any of Clauses 1-3, wherein the holder further comprises a stability extension portion provided on a proximal end of the holder.

Clause 5: The device of Clause 4, wherein the stability extension portion includes at least one retention bump that extends inwardly from an inner surface of the stability extension portion to contact the patient's finger held in the holder.

Clause 6: The device of any of Clauses 1-5, wherein at least one wing of the at least two wings includes an anti-touch protrusion that extends outwardly from an outer surface of the wings.

Clause 7: The device of Clause 6, wherein the anti-touch protrusion is positioned beneath a touch pad of the at least one wing to ensure a user grips the at least one wing at the touch pad and not beneath the touch pad.

Clause 8: The device of any of Clauses 1-7, wherein a proximal end of the holder includes a flared edge to receive the patient's finger.

Clause 9: The device of any of Clauses 1-8, wherein a transition portion is positioned between the holder and the container engagement portion to receive at least a portion of the patient's finger during use of the device.

Clause 10: The device of any of Clauses 1-9, wherein a distal end of the holder includes a curved edge to receive a patient's fingertip while allowing the patient's fingernail to extend past the distal end of the holder.

Clause 11: The device of any of Clauses 1-10, wherein the holder includes a finger receiving portion that includes at least one bulge on an outer surface of the finger receiving portion to prevent pressure on an arterial blood supply of the patient's finger held in the finger receiving portion.

Clause 12: A device for obtaining a blood sample, the device comprising: a holder for receiving a sample source, the holder having an actuation portion and a port; and a container engagement portion connected to the holder; wherein the actuation portion comprises at least two wings that are angled relative to a patient's finger held in the holder to create a pressure gradient towards a fingertip of the patient's finger.

Clause 13: The device of Clause 12, wherein the at least two wings are positioned on the holder at a position proximal of a fingernail of the patient's finger and distal from a first knuckle of the patient's finger.

Clause 14: The device of Clause 12 or 13, wherein each of the at least two wings includes a touch pad that ensures a user squeezes the wings at a desired location.

Clause 15: The device of any of Clauses 12-14, wherein the holder further comprises a stability extension portion provided on a proximal end of the holder.

Clause 16: The device of Clause 15, wherein the stability extension portion includes at least one retention bump that extends inwardly from an inner surface of the stability extension portion to contact the patient's finger held in the holder.

Clause 17: The device of any of Clauses 12-16, wherein at least one wing of the at least two wings includes an anti-touch protrusion that extends outwardly from an outer surface of the wings.

Clause 18: The device of Clause 17, wherein the anti-touch protrusion is positioned beneath a touch pad of the at least one wing to ensure a user grips the at least one wing at the touch pad and not beneath the touch pad.

Clause 19: The device of any of Clauses 12-18, wherein a proximal end of the holder includes a flared edge to receive a patient's finger.

Clause 20: The device of any of Clauses 12-19, wherein a transition portion is positioned between the holder and the container engagement portion to receive at least a portion of the patient's finger during use of the device.

Clause 21: The device of any of Clauses 12-20, wherein a distal end of the holder includes a curved edge to receive a patient's fingertip while allowing the patient's fingernail to extend past the distal end of the holder.

Clause 22: The device of any of Clauses 12-21, wherein the holder includes a finger receiving portion that includes at least one bulge on an outer surface of the finger receiving portion to prevent pressure on an arterial blood supply of the patient's finger held in the finger receiving portion.

Clause 23: A device for obtaining a blood sample, the device comprising: a holder for receiving a sample source, the holder having an actuation portion and a port; a container engagement portion connected to the holder; a collection container removably connectable to the container engagement portion, the collection container defining a collection cavity; and a lancet device removably connected to the container engagement portion, wherein the actuation portion comprises at least two wings that are angled relative to a patient's finger held in the holder to create a pressure gradient towards a fingertip of the patient's finger.

Clause 24: A method of operating a device for obtaining a blood sample, the method comprising: providing the device for operation by a user, the device comprising: a holder for receiving a sample source, the holder having an actuation portion and a port; and a container engagement portion connected to the holder; wherein the actuation portion comprises at least two wings that are angled relative to a patient's finger held in the holder to create a pressure gradient towards a fingertip of the patient's finger; squeezing the at least two wings together to create the pressure gradient on the patient's fingertip, wherein the at least two wings are squeezed between 0.25 seconds and 1 second; holding the at least two wings closed together up to 0.5 seconds; releasing pressure on the at least two wings up to 0.5 seconds; and holding open the at least two wings up to 1 second.

### BRIEF DESCRIPTION OF THE DRAWINGS

FIG. 1 is a perspective view of a holder in accordance with an embodiment of the present invention.

FIG. 2 is a top view of the holder of FIG. 1.

FIG. 3 is a side view of the holder of FIG. 1.

FIG. 4 is a front view of the holder of FIG. 1.

FIG. 5 is a rear view of the holder of FIG. 1.

FIG. 6 is a graphical illustration of a pumping method utilized by the holder of FIG. 1.

FIG. 7 is a schematic view of a transition of the holder of FIG. 1 between a passive state and an active state of the holder.

FIG. 8 is graphical illustration of blood production and discomfort measurements when using the holder of FIG. 1.

FIG. 9 is a perspective view of a device for obtaining a blood sample from a patient's finger and a collection container in accordance with another embodiment of the present disclosure.

FIG. 10 is a cross-sectional view of a device for obtaining a blood sample from a patient's finger and a lancet in accordance with another embodiment of the present disclosure.

FIG. 11 is a perspective view of a device for obtaining a blood sample from a patient's finger and a sample collection container in accordance with another embodiment of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots. The device may be used by any healthcare professionals, including nurses and doctors, or patients for self-applications when using the device.

Blood collection is fundamentally driven by pressure-driven flow. Devices or techniques either reduce the pressure outside the blood vessel (vacuum-powered flow) or increase the pressure inside the vessels. Both approaches increase the difference between the blood vessel pressure and external pressure, and increase the flow rate from inside the vessel to outside where the collection container is present. The location of squeezing can also be critical, as soft tissues (e.g. fat, skin, and musculature) are perfused with blood while hard tissues and joints are poorly perfused or are too mechanically stable to compress without patient pain.

Red blood cells (RBCs) are subject to hemolysis during collection. Hemolysis (RBC destruction) contaminates samples for diagnostic analysis, both by spilling cell contents into the liquid serum of the sample and by coloring the serum red via hemoglobin and interfering with colorimetric reactions. The amount of hemolysis during collection is driven by shear-mediated destruction of the cells due to flow rate and flow path as well as pressure-driven hemolysis where physical compression of tissues and vessels can damage cells. Hemolysis can therefore be controlled by ensuring that applied pressures and flows are not too high in any of the locations of the finger being squeezed.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

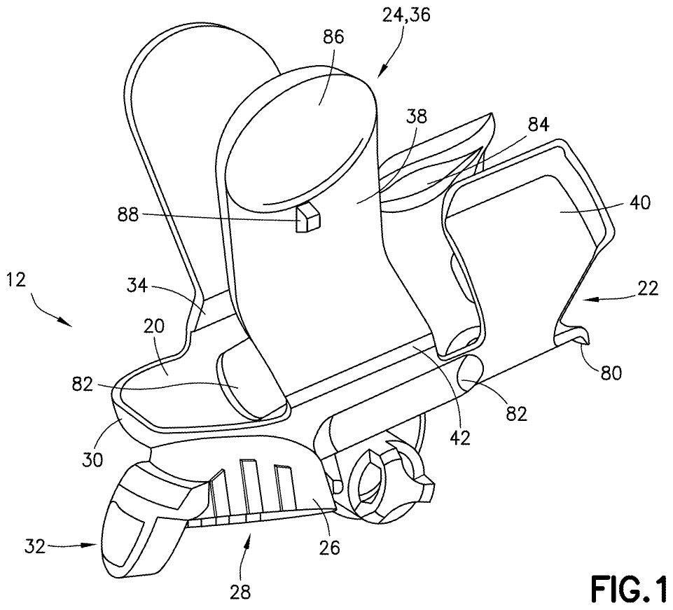

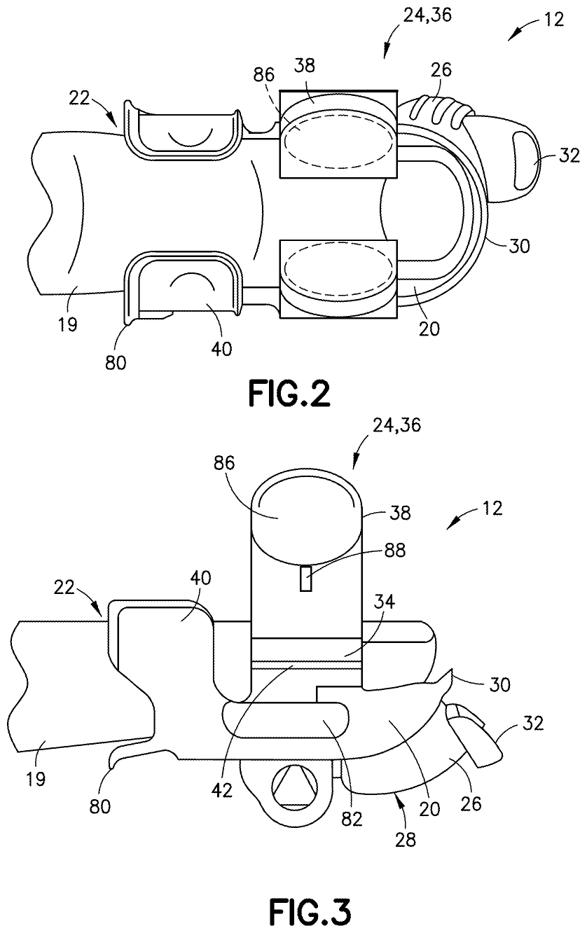

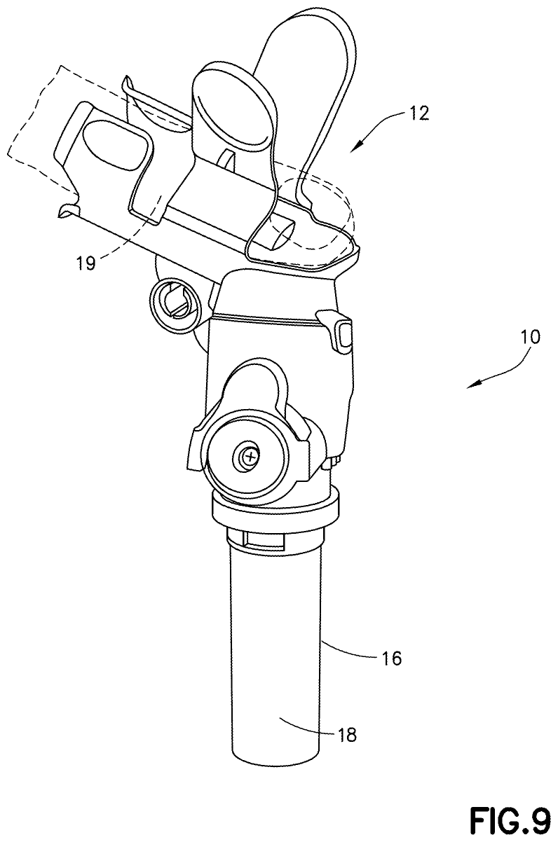

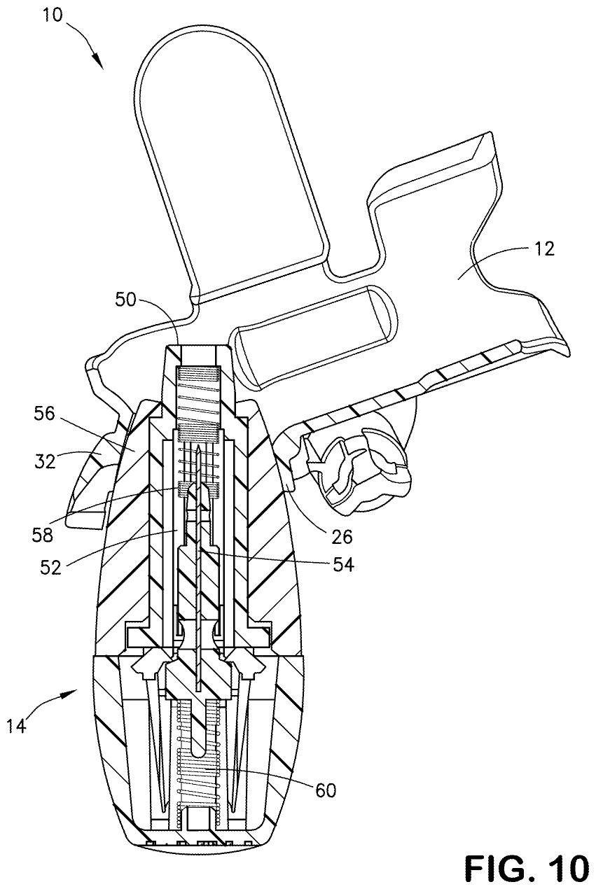

Referring to FIGS. 9 and 10, in an exemplary embodiment, a device 10 of the present disclosure includes discrete components, e.g., a holder 12 (as shown in FIGS. 1-5), a lancet housing or lancet 14, and a collection container 16. In another exemplary embodiment, a semi-integrated device of the present disclosure may include an at-angle flow and include an integrated lancet housing and collection container which can be connected with a separate holder. In another exemplary embodiment, a semi-integrated device of the present disclosure may have an in-line flow and include an integrated lancet housing and collection container which can be connected with a separate holder. In another exemplary embodiment, an integrated device of the present disclosure may have an at-angle flow and include an integrated holder, lancet housing, and collection container. In another exemplary embodiment, an integrated device of the present disclosure may have an in-line flow and include an integrated holder, lancet housing, and collection container.

Referring to FIGS. 1-5, exemplary embodiments of holders 12 of the present disclosure that are able to receive a sample source, e.g., a finger 19, for supplying a biological sample, such as a blood sample 18 (shown in FIG. 9), are shown and described. A holder 12 of the present disclosure generally includes a finger receiving portion 20 having a first opening 22 (FIG. 1), an actuation portion 24, a port 26 having a second opening 28, and a finger end guard 30. In one embodiment, the finger end guard 30 provides a stop portion for properly aligning and securing a finger 19 within the holder 12. The finger end guard 30 further assists in ensuring the patient's finger 19 is placed at a proper position within the finger receiving portion 20 so that applied pressure to the patient's finger 19 will result in adequate blood flow. In one embodiment of the present disclosure, the finger end guard 30 may have a curved fingertip rest that ensures the patient's finger 19 stops at an end of the finger receiving portion 20 while permitting the patient's finger nail to clear the end of the finger receiving portion 20. The finger receiving portion 20 permits use of the holder 12 with artificial and natural fingernail styles present in the patient population.

The first opening 22 of the finger receiving portion 20 is configured for receiving a sample source, e.g., a finger 19, for supplying a biological sample, such as a blood sample 18. It can be appreciated that the sample source could include other parts of the body capable of fitting within the first opening 22. The port 26 is in communication with the finger receiving portion 20. For example, with a finger 19 received within the holder 12, the port 26 is in communication with a portion of the finger 19. A holder 12 of the present disclosure can be sized to accommodate all finger sizes.

The second opening 28 of the port 26 is configured for receiving a lancet housing 14 and a collection container 16 as described in more detail below. In one embodiment, the port 26 includes a locking portion 32 for securely receiving the lancet housing 14 and the collection container 16 within the port 26.

In one embodiment, the actuation portion 24 is transitionable between a first position in which the holder 12 defines a first diameter and a second position which the holder 12 defines a second diameter, wherein the second diameter is less than the first diameter. In one embodiment, the actuation portion 24 is transitionable between a first position in which the holder 12 defines a first elliptical shape, and a second position in which the holder 12 defines a second elliptical shape, wherein the first elliptical shape is different than the second elliptical shape. In this manner, with the holder 12 in the second position with a reduced diameter, a portion of the holder 12 contacts the sample source and the actuation portion 24 of the holder 12 is able to pump and/or extract blood 18 as described in more detail below.

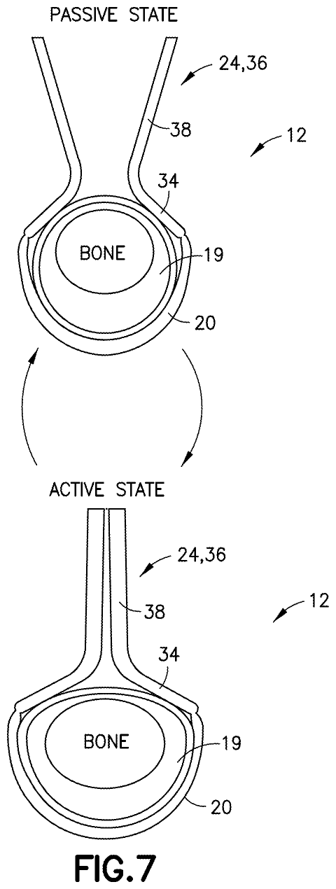

Referring to FIGS. 1, 3, and 4, in one embodiment, the actuation portion 24 includes a contact member 34. Referring to FIG. 7, with the actuation portion 24 in the first position, the contact member 34 is in a disengaged position, i.e., the contact member 34 is provided in a first position with respect to a sample source, e.g., the finger 19, such that the contact member 34 may be in slight contact therewith. Referring to FIG. 7, with the actuation portion 24 in the second position, the contact member 34 is in an engaged position, i.e., the contact member 34 is provided in a second position with respect to the sample source, e.g., the finger 19, such that the contact member 34 is in an applied pressure contact with the finer 19, and the actuation portion 24 of the holder 12 is able to pump and/or extract blood 18. For example, with the contact member 34 in the engaged position, the contact member 34 exerts a pressure on the sample source.

Referring to FIGS. 1-5, in one embodiment, the actuation portion 24 includes a pumping member 36 for applying pressure to the sample source, e.g., the finger 19. In one embodiment, the pumping member 36 comprises a pair of opposed tabs or wings 38. In such an embodiment, each tab 38 may include a contact member 34. Referring to FIGS. 1 and 3, in one embodiment, the holder 12 includes a living hinge portion 42. The living hinge portion 42 allows a user to squeeze the wings 38 between a first position (passive state) (FIG. 7) and a second position (active state) (FIG. 7). The use of the tabs or wings 38 to draw blood out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12 while flexing to accommodate the largest patient fingers within a holder 12 without blood occlusion. In one embodiment, the wings 38 may be positioned on the finger receiving portion 20 at a position located proximal of a patient's fingernail and distal of a patient's first knuckle to avoid hard tissues on the patient's finger 19.

Advantageously, the holder 12 of the present disclosure allows a user to repeatedly squeeze and release the wings 38 to pump and/or extract blood 18 from a finger 19 until a desired amount of blood 18 is filled in a collection container 16. The wings 38 are configured to flex to maintain gentle contact with a range of patient finger sizes that may be used with the holder 12 and to retain the holder 12 on the patient's finger 19. The wings 38 may also provide active pressure features for the holder 12. In one embodiment, the wings 38 may have a length, thickness, and angle optimized for ideal squeeze pressure on the patient's finger 19. In one embodiment, the location of the wings 38 on the holder 12 ensure that soft tissue of the patient's finger 19 is put under pressure while avoiding hard tissues in the patient's finger 19, such as the patient's knuckles. Further, in one embodiment, an angle between the wings 19 and patient's finger 19 tapers to create a pressure gradient towards the patient's fingertip to aid in blood flow from the patient's finger 19. The angle of the wings 38 relative to the natural taper of the finger 19 creates a small pressure gradient from distal to proximal end of the finger 19, ensuring the direction of flow is towards the fingertip while avoiding finger milking per international best practices for sample quality.

In one embodiment, the wings 38 may also include touch pads 86 on an outer surface thereof to be gripped by the user of the holder 12. The touch pads 86 may ensure that users squeeze the wings 38 at the correct desired location for proper squeeze pressure, comfort, and hemolysis when drawing blood from the patient's finger 19. In one embodiment, the wings 38 may also include anti-touch protrusions 88 that extend from an outer surface of the wings 38. In one embodiment, the anti-touch protrusions 88 are positioned on the wings 38 beneath the touch pads 86. The anti-touch protrusions 88 extend outwardly from the wings 38 and are positioned and configured to discourage users from squeezing too low on the wings 38 and applying too much pressure to the wings 38. The anti-touch protrusions 88 ensure the user does not grip the wings 38 too low to allow the wings 38 to flex as needed for proper pressure application. Users may be instructed to know that, once the anti-touch protrusions 88 are contacted, the user's grip must be moved upwards closer to the touch pads 86 to ensure proper pressure is being applied to the patient's finger 19.

Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Referring to FIG. 1, in one embodiment, the holder 12 includes a stability extension portion 40. This provides additional support for the holder 12 to be securely placed onto a finger 19. In one embodiment, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

In one embodiment, the finger receiving portion 20 is formed of a flexible material. In some embodiments, the finger receiving portion 20 and the port 26 are formed from a flexible material.

A device 10 for obtaining a blood sample 18 of the present disclosure includes a lancet housing or lancet 14 that is removably connectable to a port 26 of a holder 12. Referring to FIG. 10, in one embodiment, the lancet housing 14 includes an inlet or opening 50, an interior 52, a puncturing element 54, an engagement portion 56, a retractable mechanism 58, and a drive spring 60. In one embodiment, the puncturing element 54 is moveable between a pre-actuated position wherein the puncturing element 54 is retained within the interior 52 of the lancet housing 14 and a puncturing position wherein at least a portion of the puncturing element 54 extends through the inlet 50 of the lancet housing 14 to lance a portion of a finger 19.

In one embodiment, the lancet 14 of the present disclosure is a contact activated lancet and may be constructed in accordance with the features disclosed in U.S. Patent Application Publication No. 2006/0052809 filed May 6, 2005, entitled “Contact Activated Lancet Device”, and commonly assigned with the present application, the entire disclosure of which is hereby expressly incorporated herein by reference thereto.

In one embodiment, the lancet housing 14 may be a separate component from the holder 12 and the collection container 16. In some embodiments, the collection container 16 and the lancet housing 14 form a single component that is removably connectable to the port 26 of the holder 12. In some embodiments, the collection container 16, the lancet housing 14, and the holder 12 form a single component.

Referring to FIG. 10, in one embodiment, with the holder 12 and the lancet housing 14 being separate components, the lancet housing 14 is removably connectable to the port 26 of the holder 12. In such an embodiment, the lancet housing 14 includes an engagement portion 56. Referring to FIG. 10, in one embodiment, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. In some embodiments, the port 26 of the holder 12 includes a plurality of ribs for securing and locking the lancet 14 or the collection container 16 in the port 26.

To activate the lancet 14, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

In one embodiment, the lancet 14 includes a drive spring 60 disposed within the interior 52 of the lancet housing 14 for biasing the puncturing element 54 toward the puncturing position. After puncturing, the puncturing element 54 is immediately retracted and safely secured within the interior 52 of the lancet housing 14.

In one embodiment, the lancet 14 of the present disclosure is used to lance the skin of a finger 19 and then a blood sample 18 is squeezed into a collection container 16 as described in more detail below.

In one embodiment, the lancet housing 14 of the present disclosure is used to lance the skin of a finger 19 along a lance path and then a blood sample 18 flows down a blood flow path at an angle to the lance path as described in more detail below.

In one embodiment, the lancet 14 can include a hollow needle. In such an embodiment, the lancet housing 14 of the present disclosure is used to lance the skin of a finger 19 along a lance path and then a blood sample 18 flows along a parallel blood flow path through the hollow needle.

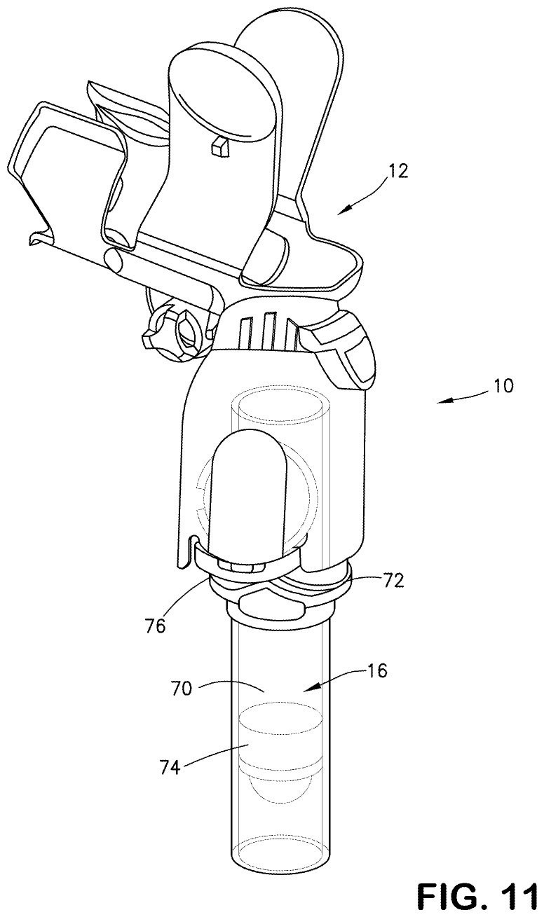

As shown in FIGS. 9 and 11, a device 10 for obtaining a blood sample 18 of the present disclosure includes a collection container 16 that is removably connectable to the port 26 (shown in FIG. 1) of the holder 12. With specific reference to FIG. 11, the collection container 16 defines a collection cavity 70 for receiving a blood sample 18 (shown in FIG. 9), a container engagement portion 72, a blood collector portion 74, and a cap or septum 76. Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

In one embodiment, the collection container 16 may be a separate component from the holder 12 and the lancet housing 14. In some embodiments, the collection container 16 and the lancet housing 14 form a single component that is removably connectable to the port 26 of the holder 12. In some embodiments, the collection container 16, the lancet housing 14, and the holder 12 form a single component.

In one embodiment, with the holder 12 and the collection container 16 being separate components, the container 16 is removably connectable to the port 26 of the holder 12. In such an embodiment, the container 16 includes a container engagement portion 72. In one embodiment, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

It can be appreciated that several types of collection containers 16 can be used with the device 10 of the present disclosure. It can also be appreciated that the collection container 16 can be associated with a separate dispensing unit or the collection container 16 can include an integral dispensing portion for dispensing the blood 18 to a testing device.

Referring to FIGS. 1-5, 9, and 10, use of a device 10 of the present disclosure having discrete components, e.g., a holder 12, a lancet housing or lancet 14, and a collection container 16, will now be described.

Referring to FIG. 3, first a desired finger 19 is cleaned and a holder 12 having an appropriate size for the desired finger 19 is selected and placed onto the finger 19 securely. Next, referring to FIG. 10, a lancet housing 14 is connected to the port 26 of the holder 12. As discussed above, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. With the lancet 14 connected to the port 26 of the holder 12, the lancet is in communication with the finger 19.

When it is desired to activate the lancet 14 to lance the skin of a finger 19, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

After the finger 19 is lanced to create blood 18 flow from the finger 19, the lancet 14 is removed from the holder 12 and the collection container 16 is pushed into the port 26 of the holder 12. Referring to FIG. 11, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

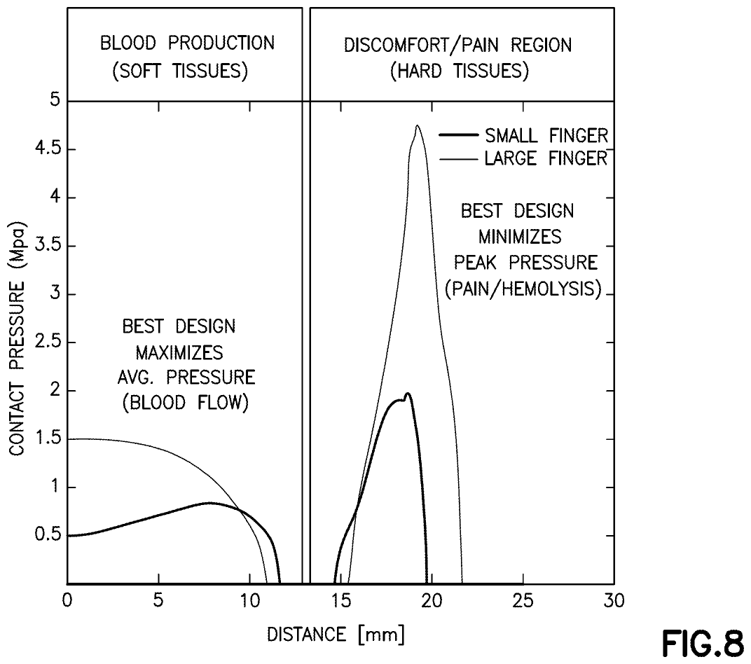

Referring to FIGS. 7 and 8, with the container 16 properly secured to the holder 12 for collection of a blood sample 18, a user is able to repeatedly squeeze and release the wings 38 of the holder 12 to pump and/or extract blood 18 from a finger 19 until a desired amount of blood 18 is filled in a collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

For example, referring to FIGS. 7 and 8, in one embodiment, the actuation portion 24 includes a contact member 34. Referring to FIG. 7, with the actuation portion 24 in the first position, the contact member 34 is in a disengaged position, i.e., the contact member 34 is in the first position with respect to the sample source, e.g., the finger 19. Referring to FIG. 7, with the actuation portion 24 in the second position, the contact member 34 is in an engaged position, i.e., the contact member 34 is in the second position and in applied pressure contact with a sample source, e.g., the finger 19, and the actuation portion 24 of the holder 12 is able to pump and/or extract blood 18. For example, with the contact member 34 in the engaged position, the contact member 34 exerts a pressure on the sample source.

Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

The devices of the present disclosure are compatible with any known testing device, whether the testing device is off-site or a point-of-care testing device. Various point-of-care testing devices are known in the art. Such point-of-care testing devices include test strips, glass slides, diagnostic cartridges, or other testing devices for testing and analysis. Test strips, glass slides, and diagnostic cartridges are point-of-care testing devices that receive a blood sample and test that blood for one or more physiological and biochemical states. There are many point-of-care devices that use cartridge based architecture to analyze very small amounts of blood bedside without the need to send the sample to a lab for analysis. This saves time in getting results over the long run, but creates a different set of challenges versus the highly routine lab environment. Examples of such testing cartridges include the i-STAT® testing cartridge from the Abbot group of companies. Testing cartridges such as the i-STAT® cartridges may be used to test for a variety of conditions including the presence of chemicals and electrolytes, hematology, blood gas concentrations, coagulation, or cardiac markers. The results of tests using such cartridges are quickly provided to the clinician.

The collection container 16 may also contain a sample stabilizer, e.g., an anticoagulant, to stabilize a blood sample and/or a component of a blood sample disposed therein. The collection container 16 may also include at least one fill line(s) corresponding to a predetermined volume of sample. The collection container may also indicate/meter a collected volume of blood.

In another exemplary embodiment, a device for obtaining a blood sample 18 of the present disclosure has an at-angle flow and includes an integrated holder 12, lancet housing 14, and collection container 16. In such an embodiment, a user does not have to connect a separate lancet housing 14 to the port 26 of the holder 12, remove the lancet 14 after lancing the skin of a finger 19, and then connect a collection container 16 to the port 26 of the holder 12. Instead, the lancet housing 14 is permanently secured within the port 26 of the holder 12. The lancet housing 14 includes a blood flow channel. The collection container 16 is secured to the lancet housing 14 and includes a blood collector portion 74 that is removably connectable to a portion of the lancet housing 14.

In one embodiment, with the container 16 connected to the lancet housing 14, the longitudinal axis of the lancet housing 14 is at an angle to the longitudinal axis 104 of the container 16. In one embodiment, the lancet housing 14 is used to lance the skin of a finger 19 along a lance path and then a blood sample 18 flows down a blood flow path at an angle to the lance path.

Any of the devices for obtaining a blood sample of the present disclosure can be used as a self-standing disposable device and/or in association with an external power source for pain reduction control. For example, a portion of holder 12 may include embedded electrodes which receive a signal from an external pain control module to deliver at least one of heat, vibration, or transcutaneous electrical nerve stimulation (TENS) for pain reduction control. The devices for obtaining a blood sample of the present disclosure may also include various options for on-board plasma separation. The devices for obtaining a blood sample of the present disclosure may also include a unique sample identifier that can be paired with patient information at the time of collection. The devices for obtaining a blood sample of the present disclosure may also include on-board diagnostic feedback at the time of collection. A device for obtaining a blood sample of the present disclosure may also allow for dual collection, e.g., the collection of two samples into two separate containers, using multiple collection ports which enable the collection of multiple samples from the same source and treating the samples with different sample stabilizers, such as anticoagulants.

A device for obtaining a blood sample of the present disclosure significantly simplifies and de-skills large volume capillary collection from a finger relative to the conventional capillary collection using lancet and capillary tube. The devices of the present disclosure eliminate blood exposure and prevents device reuse.

The devices for obtaining a blood sample of the present disclosure simplify, deskill, and streamline the collection process. This is all achieved by a self-contained closed system device which after it is placed onto a finger will provide lancing, blood extraction, stabilization, and containment functions, all in one unit.

The devices for obtaining a blood sample of the present disclosure may be associated with a self-standing unit that provides automated pumping, controlled finger squeezing, and automated sample labeling and processing.

With reference to FIGS. 1-5, according to various embodiments of the present disclosure, additional features of the holder 12 are shown and described. In one embodiment, the holder 12 may include passive pressure features that assist in drawing a blood sample 18 from the patient's finger 19. The passive pressure features are designed to aid and ensure proper positioning of the holder 12 before and during use of the holder 12 for blood collection without restricting blood flow. The holder 12 may include a flared edge 80 provided on the opening 22 of the finger-receiving portion 20. The flared edge 80 may create a comfortable position for the patient's finger 19 to rest upon instead of a sharp corner. Instead, the flared edge 80 may include a rounded or curved edge that assists in providing a comfortable resting space for the patient's finger 19.

In one embodiment, the holder 12 may include at least two bulges 82 that extend from an outer surface of the finger receiving portion 20. The bulges 82 may have rounded edges and may be configured to prevent pressure on the arterial blood supply of the patient's finger 19 that is positioned in the finger receiving portion 20.

In one embodiment, holder 12 may also include at least one retention bump 84 provided on the stability extension portion 40. In one embodiment, the stability extension portion 40 includes two retention bumps 84. The retention bump 84 may extend from an inner surface of the stability extension portion 40 to engage and contact the patient's finger 19 when positioned in the finger receiving portion 20. The retention bumps 84 may have a curved surface so as to provide a comfortable engagement with the patient's finger 19. The retention bumps 84 may be provided to secure the holder 12 to the patient's finger 19 without restricting arterial blood supply to the patient's finger 19.

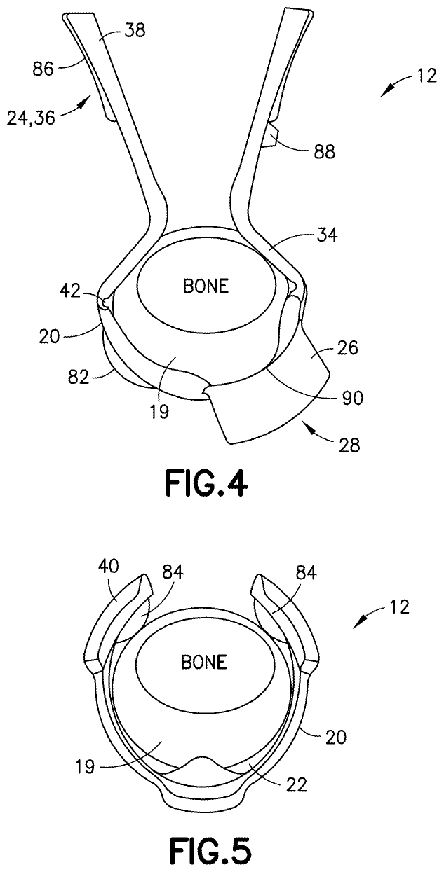

In one embodiment, as shown in FIG. 4, a transition 90 from the finger receiving portion 20 to the opening 28 allows for expansion of the patient's finger 19 under pressure during squeezing of the wings 38 without a flow restriction or hemolysis. The transition 90 permits a portion of the patient's finger 19 to extend into the opening 28 to allow the finger to expand under pressure in the finger receiving portion 20. By permitting this expansion of the patient's finger 19, a restriction of the blood flow from the patient's finger 19 and hemolysis is avoided.

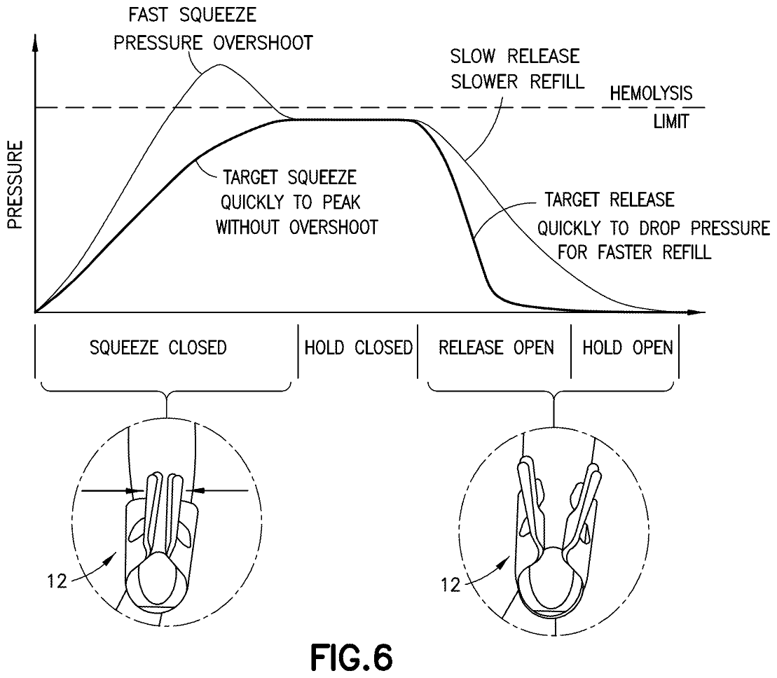

With reference to FIG. 6, according to one embodiment of the present disclosure, a method of using the holder 12 to draw blood from a patient's finger 19 is described. The method of use of the holder 12 has been described above in detail, while the following disclosure describes a specific squeezing rhythm for using the holder 12 to ensure an adequate and desired volume of blood is drawn from the patient's finger 19. It is to be understood, however, that any number of squeezing rhythms can be used with the holder 12 including faster, slower, more varied, and different pressured rhythms. In one embodiment, the squeezing rhythm first includes squeezing the wings 38 of the holder 12 towards one another to apply pressure to the patient's finger 19. In one embodiment, the wings 38 are squeezed a minimum 0.25 seconds and a maximum of 1 second to close the wings 38 towards one another. In one embodiment, the wings 38 are then held closed with one another. The wings 38 may be held together in the closed position for up to 0.5 seconds. In one embodiment, the wings 38 may not be held in the closed position and, instead, are squeezed closed and then released open. In one embodiment, the wings 38 may be released open in 0.5 seconds. The wings 38 may then be held in the open position for up to 1 second. It is also contemplated that the wings 38 may not be allowed to rest in the open position and, instead, are squeezed closed again after being released from the closed position.

As shown in FIG. 6, according to one embodiment of the present disclosure, a squeezing rhythm for the holder 12 is shown and described. The wings 38 may be squeezed closed and released to an open position in a pumping manner. It is also contemplated that the wings 38 may be held in the closed position for a duration of time before being released to the open position. The wings 38 move between a state of passive pressure, which includes the resting fit of the holder 12 on the finger 19 which allows the holder 12 to stay firmly in place without restricting blood flow, and an active pressure, which includes the pressure applied by the user and the holder 12 to cause blood to flow from the finger 19 without hemolyzing or compromising the blood sample. In one embodiment of the present disclosure, it is desired that the user apply a predetermined desired pressure to the wings 38 to quickly peak to the desired pressure without an overshoot in pressure. An overshoot in pressure is realized by squeezing the wings 38 of the holder 12 too quickly. Therefore, a more uniform pressure applied to the wings 38 is desired to avoid an overshoot in pressure, which may result in an undesired blood flow from the patient's finger 19. Further shown in FIG. 6, a slow release of the wings 38 is typically not desired as it creates a slower refill for the blood flow in the patient's finger 19. Instead, a quicker release of the wings 38 is desired to quickly drop pressure on the patient's finger 19 to allow for a faster refill of blood flow through the patient's finger 19. The passive and active pressure features, and the rhythm of moving between the active squeezed state and passive rest state, work together as a system to control how and where the finger 19 is squeezed to provide blood flow rates equivalent to expert capillary collection healthcare professionals at less than 25% of the average hemolysis.

With reference to FIGS. 7 and 8, the different states or positions of the holder 12 are shown to detail blood flow and discomfort in relation to the pressure applied to the patient's finger 19. The graphical illustration in FIG. 8 illustrates a contact pressure in relation to blood production achieved from the patient's finger 19 and the eventual discomfort and pain experienced by the patient upon extended pressure. The graphical illustration charts the pressure applied around the circumference of the patient's finger during a squeeze of the holder 12. The graphical illustration shows that the further around the circumference of the patient's finger 19 from a bottom dermis portion of the patient's finger 19 the pressure is applied, the more pain and discomfort is experienced by the patient, especially in the hard tissue areas of the finger 19.

The present holder 12 provides advantages over conventional capillary blood collection devices. The holder 12 is configured to align with a patient's finger 19 features, ensuring that the holder 12 consistently and securely remains in place and applies pressure in the correct location. This feature was accomplished by analyzing several sources of anatomical information (finger width and length, knuckle and artery locations) to limit squeezing to soft tissues near the collection site while avoiding pressure on hard tissues or blood vessels. Further, the wings 38 are configured to apply pressure in two stages. The first stage has pressure on the finger increased proportionally to the applied pressure. However, as intensity increases, the wings 38 begin to flex and bend until they touch and cannot displace any further. This allows enough pressure to have adequate blood flow but limits maximum pressure to avoid hemolysis. This avoids issues with health care workers squeezing harder to promote flow and vacuum methods applying too little pressure. The prescribed squeezing rhythm is also prescribed to allow blood to refill the fingertip. This avoids user variation from manual techniques and consistent pressure by vacuum methods which do not allow capillary beds to be replenished.

The finger-squeezing wings 38 further define the pressure location and can only be operated one way. The wings 38 also have the pads 86 to promote proper finger placement and operation by workers and anti-touch features to avoid improper operation. This prevents the use of the improper “milking” technique and ensures correct operation of the wings 38, which both contribute to better hemolysis. The wings 38 further act as levers, meaning users apply a lower peak force to close the wings 38 and instead travel further during the squeezing motion than squeezing the finger 19 directly. This turns manual squeezing from a small displacement, high force motion which is prone to fatigue to a larger displacement, lower force motion more suitable to routine use.

The device was designed to apply pressure to the soft tissues of the fingertip, limit the peak pressure applied spatially on the finger, and maximize the average pressure applied across the tissue within empirically determined hemolysis limits. Limiting applied pressure to soft-tissues lowered the maximum force required to generate blood. Reducing the peak pressure applied to individual regions of the finger (e.g. side, top, bottom) would contribute to hemolysis or patient pain. Maximizing average pressure ensured that blood flow would still be adequate to have practical capillary blood collection.

The wings 38 also limit user fatigue and maximum pressure applied by acting as deformable levers. Levers typically function by exchanging force for displacement: users must squeeze across a longer travel distance but the force required is much lower than squeezing the finger directly. This reduces user fatigue which is typically driven by peak force generated.

However, the wings 38 are also designed to flex. When force is initially applied to the wings 38, the soft tissues of the finger compress and squeeze blood out like a sponge. As blood leaves and the soft tissues compress further, they stiffen and require more force to continue squeezing. This regime of squeezing has diminished value for blood flow but can contribute greatly to hemolysis as pressure increases rapidly. As this occurs, the force applied on the wings 38 increases proportionally. In this regime the wings 38 begin to act as a classic cantilever beam under a point load—the ends of the wing 38 begin deflecting in addition to the angular displacement of the wing 38. This causes the wings 38 to quickly touch each other and limit the total amount of displacement, thus limiting the maximum pressure applied more than geometrically stiff, non-flexing wings. The wings 38 have the added benefit of only deflecting when soft tissues have been compressed enough, that is, only when blood has been adequately squeezed from the finger and further squeeze pressure would be detrimental.

According to one embodiment of the present disclosure, the patient's finger 19 and/or the holder 12 may be disinfected before use of the device 10 and the holder 12. The goal of cleaning/disinfection is to minimize the risk of infection or irritation of the collection site, following skin puncture and blood collection. The patient's ring finger or middle finger may be the puncture site for the lancet 14. Therefore, before puncturing the patient's finger 19 and/or inserting the patient's finger 19 into the holder 12, the patient's finger 19 may be disinfected with an alcohol wipe. In some examples, the collection site of the patient's finger, the proximal tissues such as the adjacent fingers and the rest of the patient's hand, and/or at least the puncture site and the palm of the patient's hand with as much of the surround area may be disinfected with the alcohol wipe. 70% Isopropyl alcohol and water may be used to disinfect the patient's finger 19 and the holder 12, or 70% ethyl alcohol and water may be used to disinfect the patient's finger 19 and the holder 12. The alcohol may be permitted to dry for at least 10 seconds to ensure evaporation. Complete drying is necessary to prevent additional pain during lancing and avoid additional sample hemolysis leading to reduced sample quality.

In another embodiment of the present disclosure, the holder 12 may be disinfected with an alcohol wipe. In particular, a 70% alcohol wipe may be used to disinfect the holder. The finger receiving portion 20, the actuation portion 24, and any other remaining portions of the holder 12 may be wiped with the alcohol wipe to disinfect the surfaces of the holder 12. The disinfection of the patient's finger 19 and the holder 12 assists in preventing introduction or reintroduction of contaminants into or onto patient's finger 19. Since the puncture site of the patient's finger 19 will rest in the opening 28 of the holder, the disinfected portion of the patient's finger 19 will have no need to contact a surface of the holder 12, thereby further assisting in preventing contamination of the patient's finger 19. In another embodiment of the present disclosure, instead of using an alcohol wipe, a swap and a 70% alcohol solution dispenser may be used to disinfect the patient's finger 19 and/or the holder 12. In particular, the swap may be used to wipe the surfaces of the finger receiving portion 20 and other portions of the holder 12 until there is a sheen on the surfaces of the holder 12 to indicate that the holder 12 has been properly disinfected.

Capillary collection blood draws are typically performed by health care workers either using their fingers to manually squeeze the tissue around the puncture site or by a device using vacuum pressure to pull blood from the site. However, capillary blood collection is an uncommon blood collection technique for the purposes of core lab panel testing especially in adults due to volume and sample quality issues. Venipuncture and venous blood are the gold standard, with capillary blood samples only used as a substitute when a venous sample cannot be acquired. The classic example is neonatal blood testing where large accessible veins are not available for blood collection via venipuncture. Capillary blood would be acquired in this case using a lancet, targeting the heel of the infant. Prior to capillary collection via the heel-stick method, the heel is first cleaned. This is accomplished most commonly using an alcohol wipe and gauze. However, collection occurs directly into a capillary tube and does not utilize a Yoda finger cuff or analogous device to aid the health care worker during sample collection, and therefore no device cleansing step exists.

Previous art/practice utilizes an alcohol wipe to disinfect the collection site followed immediately by sample collection, which is true for venipuncture and conventional capillary collection. However, the immediate advancement to collection can lead to additional pain experienced by the patient due to the alcohol sting, but more importantly can also impact sample quality via increased hemolysis. This issue may be addressed by wiping the wet alcohol spot with a gauze, which effectively dries the area but can reintroduce contaminants and elevate the risk of infection.

Disinfection and cleansing of the puncture site acts to reduce the bacterial load and minimize the risk of nosocomial infections. The use of alcohols (ethanol or isopropanol) act by dehydrating microorganisms and denaturing proteins when in direct contact. The solutions (generally 70% alcohol) are also quite volatile and as a result only requires a short evaporation period before the disinfected site is free for blood collection. These features make for a great disinfection solution, leaving no residue or artifact behind if proper directions are followed.

While an embodiment of a capillary blood collection device is shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A device for obtaining a blood sample, the device comprising:
a holder for receiving a sample source, the holder having an actuation portion and a port;
a container engagement portion connected to the holder; and
a collection container removably connectable to the container engagement portion, the container defining a collection cavity,
wherein the actuation portion comprises at least two wings that are angled relative to a patient's finger held in the holder to create a pressure gradient towards a fingertip of the patient's finger.

2. The device of claim 1, wherein the at least two wings are positioned on the holder at a position proximal of a fingernail of the patient's finger and distal from a first knuckle of the patient's finger.

3. The device of claim 1, wherein each of the at least two wings includes a touch pad that ensures a user squeezes the wings at a desired location.

4. The device of claim 1, wherein the holder further comprises a stability extension portion provided on a proximal end of the holder.

5. The device of claim 4, wherein the stability extension portion includes at least one retention bump that extends inwardly from an inner surface of the stability extension portion to contact the patient's finger held in the holder.

6. The device of claim 1, wherein at least one wing of the at least two wings includes an anti-touch protrusion that extends outwardly from an outer surface of the wings.

7. The device of claim 6, wherein the anti-touch protrusion is positioned beneath a touch pad of the at least one wing to ensure a user grips the at least one wing at the touch pad and not beneath the touch pad.

8. The device of claim 1, wherein a proximal end of the holder includes a flared edge to receive the patient's finger.

9. The device of claim 1, wherein a transition portion is positioned between the holder and the container engagement portion to receive at least a portion of the patient's finger during use of the device.

10. The device of claim 1, wherein a distal end of the holder includes a curved edge to receive a patient's fingertip while allowing the patient's fingernail to extend past the distal end of the holder.

11. The device of claim 1, wherein the holder includes a finger receiving portion that includes at least one bulge on an outer surface of the finger receiving portion to prevent pressure on an arterial blood supply of the patient's finger held in the finger receiving portion.

12. A device for obtaining a blood sample, the device comprising:
a holder for receiving a sample source, the holder having an actuation portion and a port; and
a container engagement portion connected to the holder;
wherein the actuation portion comprises at least two wings that are angled relative to a patient's finger held in the holder to create a pressure gradient towards a fingertip of the patient's finger.

13. The device of claim 12, wherein the at least two wings are positioned on the holder at a position proximal of a fingernail of the patient's finger and distal from a first knuckle of the patient's finger.

14. The device of claim 12, wherein each of the at least two wings includes a touch pad that ensures a user squeezes the wings at a desired location.

15. The device of claim 12, wherein the holder further comprises a stability extension portion provided on a proximal end of the holder.

16. The device of claim 15, wherein the stability extension portion includes at least one retention bump that extends inwardly from an inner surface of the stability extension portion to contact the patient's finger held in the holder.

17. The device of claim 12, wherein at least one wing of the at least two wings includes an anti-touch protrusion that extends outwardly from an outer surface of the wings.

18. The device of claim 17, wherein the anti-touch protrusion is positioned beneath a touch pad of the at least one wing to ensure a user grips the at least one wing at the touch pad and not beneath the touch pad.

19. The device of claim 12, wherein a proximal end of the holder includes a flared edge to receive a patient's finger.

20. The device of claim 12, wherein a transition portion is positioned between the holder and the container engagement portion to receive at least a portion of the patient's finger during use of the device.

21. The device of claim 12, wherein a distal end of the holder includes a curved edge to receive a patient's fingertip while allowing the patient's fingernail to extend past the distal end of the holder.

22. The device of claim 12, wherein the holder includes a finger receiving portion that includes at least one bulge on an outer surface of the finger receiving portion to prevent pressure on an arterial blood supply of the patient's finger held in the finger receiving portion.

23. A device for obtaining a blood sample, the device comprising:
a holder for receiving a sample source, the holder having an actuation portion and a port;
a container engagement portion connected to the holder;
a collection container removably connectable to the container engagement portion, the collection container defining a collection cavity; and
a lancet device removably connected to the container engagement portion,
wherein the actuation portion comprises at least two wings that are angled relative to a patient's finger held in the holder to create a pressure gradient towards a fingertip of the patient's finger.

24. (canceled)
